(19) United States
(12) Patent Application Publication
(10) Pub. No.: US 2023/0235288 A1
(43) Pub. Date: Jul. 27, 2023

(54) PERIPHERAL BLOOD DERIVED SMALL PLURIPOTENT CELLS

(71) Applicant: Tithon Biotech, Inc., San Diego, CA (US)

(72) Inventor: Vasilis Paspaliaris, San Diego, CA (US)

(21) Appl. No.: 18/002,151

(22) PCT Filed: Jun. 21, 2021
(86) PCT No.: PCT/US2021/038288

§ 371 Date: Dec. 16, 2022

Related U.S. Application Data
(60) Provisional application No. 63/042,944, filed on Jun. 23, 2020.
(60) Provisional application No. 63/070,164, filed on Aug. 25, 2020.
(60) Provisional application No. 63/147,672, filed on Feb. 9, 2021.

(51) Int. Cl. C12N 5/0789

(52) U.S. Cl. CPC C12N 5/0647

(57) ABSTRACT

The present disclosure relates to populations of small pluripotent stem cells derived from peripheral blood, such as human peripheral blood. In some aspects, these small pluripotent stem cells are smaller than known stem cells and express a range of embryonic, hematopoietic, or mesenchymal stem cell markers. Also disclosed herein are methods of isolation and cryopreservation of these populations of small pluripotent stem cells. These small pluripotent stem cells may be differentiated in a wide range of cell types, which can be used in various applications such as the study of cell activity or for treatment of diseases and personalized medicine.

### CROSS-REFERENCE TO RELATED APPLICATIONS

This application claims the benefit of priority to U.S. Provisional Pat. Application No. 63/042,944, filed Jun. 23, 2020, U.S. Provisional Pat. Application No. 63/070,164, filed Aug. 25, 2020, and U.S. Provisional Pat. Application No. 63/147,672, filed Feb. 9, 2021, each of which is hereby expressly incorporated by reference in its entirety.

### FIELD

Aspects of the present disclosure relate generally to populations of small pluripotent stem cells derived from peripheral blood. Additional aspects relate to methods of purifying and methods of cryopreserving the populations of small pluripotent stem cells.

### BACKGROUND

Pluripotent stem cells are key players in regenerative medicine and the development of therapeutic approaches for tissue repair. Embryonic pluripotent stem cells were potentially excellent candidates, but have substantial limitations, including inadequate availability and ethical dilemmas for their isolation and use. The need for new stem cell populations and improved isolation and storage procedures is manifest.

### SUMMARY

The methods described herein relate to the discovery that small blood stem cells (SBSCs) that express pluripotent embryonic markers can be isolated from peripheral blood by centrifugation of the plasma fraction and filtering the resultant cell population recovered from the cell pellet in a manner, which allows the SBSCs to pass through the filter but excludes other cells larger than the SBSCs, e.g., by using a filter having a 5 µm exclusion such that cells larger than 5 µm do not pass through the filter. Once isolated, the population of cells comprising the SBSCs can be cryopreserved by resuspending the SBSCs in a cryopreservation medium, freezing the SBSCs at -80° C. and transferring the frozen SBSCs to -150° C.

Preferred embodiments provided herein include the following numbered alternatives:

1. A method of isolating small blood stem cells (SBSCs) from peripheral blood, comprising:
centrifuging the peripheral blood to isolate plasma from the peripheral blood;
centrifuging the plasma to isolate a population of cells comprising the SBSCs, preferably in a cell pellet;
resuspending the population of cells comprising the SBSCs in a liquid; and
filtering the resuspended population of cells comprising the SBSCs through a filter having a pore size, which excludes cells that are larger than the SBSCs;
thereby isolating the SBSCs.

2. The method of alternative 1, wherein the peripheral blood is centrifuged at a speed that is 400×g, 450×g, 500×g, 550×g, 600×g, 650×g, 700×g, 750×g, or 800×g, or about 400×g, about 450×g, about 500×g, about 550×g, about 600×g, about 650×g, about 700×g, about 750×g, or about 800×g, or any speed within a range defined by any two of the aforementioned speeds.

3. The method of alternative 1 or 2, wherein the plasma is centrifuged at a speed that is 1000×g, 1050×g, 1100×g, 1150×g, 1200×g, 1250×g, 1300×g, 1350×g, or 1400×g, or about 1000×g, about 1050×g, about 1100×g, about 1150×g, about 1200×g, about 1250×g, about 1300×g, about 1350×g, or about 1400×g, or any speed within a range defined by any two of the aforementioned speeds.

4. The method of any one of the preceding alternatives, wherein the peripheral blood is centrifuged with a density gradient.

5. The method of any one of the preceding alternatives, wherein the peripheral blood is centrifuged without a density gradient.

6. The method of any one of the preceding alternatives, wherein the plasma is centrifuged with a density gradient.

7. The method of any one of the preceding alternatives, wherein the plasma is centrifuged without a density gradient.

8. The method of any one of the preceding alternatives, wherein the pore size of the filter is 5 µm and cells that are larger than 5 µm are excluded.

9. The method of any one of the preceding alternatives, wherein the pore size of the filter is larger than 5 µm.

10. The method of any one of the preceding alternatives, wherein the pore size of the filter is 1 µm, 2 µm, 3 µm, 4 µm, 5 µm, 6 µm, 7 µm, 8 µm, 9 µm, 10 µm, 11 µm, 12 µm, 13 µm, 14 µm, 15 µm, 16 µm, 17 µm, 18 µm, 19 µm, or 20 µm, or about 1 µm, about 2 µm, about 3 µm, about 4 µm, about 5 µm, about 6 µm, about 7 µm, about 8 µm, about 9 µm, about 10 µm, about 11 µm, about 12 µm, about 13 µm, about 14 µm, about 15 µm, about 16 µm, about 17 µm, about 18 µm, about 19 µm, or about 20 µm, or any size within a range defined by any two of the aforementioned sizes.

11. The method of any one of the preceding alternatives, wherein the population of cells comprising the SBSCs is resuspended in an isotonic solution.

12. The method of alternative 11, wherein the isotonic solution is growth medium, 0.9% saline, 5% dextrose solution, Ringer’s lactate solution, or Ringer’s acetate solution, or any combination thereof.

13. The method of any one of the preceding alternatives, wherein the peripheral blood is mixed with an anticoagulant prior to centrifuging.

14. The method of alternative 13, wherein the anticoagulant is sodium citrate.

15. The method of any one of the previous alternatives, wherein the population of cells comprising the SBSCs comprise cells having pluripotency markers, mesenchymal stem cell markers, or hematopoietic stem cell markers, or any combination thereof.

16. The method of alternative 15, wherein the pluripotency markers comprise Nanog, Oct4, SOX-2, CXCR4, cMyc, KLF4, SSEA-3, or SSEA-4, or any combination thereof.

17. The method of alternative 15 or 16, wherein the mesenchymal stem cell markers comprise CD29, PTH1R, CD105, or CD106, or any combination thereof.

18. The method of any one of alternatives 15-17, wherein the hematopoietic stem cell markers comprise CD90, CD133, or CD45, or any combination thereof.

19. The method of any one of alternatives 15-18, wherein the SBSCs stain with Kyoto Probe 1.

20. The SBSCs isolated according to any one of the preceding alternatives.

21. A method of cryopreserving isolated SBSCs, comprising:
resuspending the SBSCs in a cryopreservation medium;
freezing the SBSCs at -80° C.; and
transferring the frozen SBSCs to -150° C.

22. The method of alternative 21, wherein the cryopreservation medium is 10:1 human serum:DMSO, mFreSR, or Bambanker.

23. A method of differentiating SBSCs to osteoblasts, comprising contacting the SBSCs with one or more compounds sufficient to differentiate pluripotent stem cells to osteoblasts, such as Purmorphamine, a 2,6,9-trisubstituted purine compound, ascorbic acid, β-glycerophosphate, and/or dexamethasone/retinoic acid or by co-culture with fetal murine osteoblasts for a time that is sufficient to differentiate the SBSCs to osteoblasts.

24. A method of differentiating SBSCs to chondrocytes, comprising contacting the SBSCs with one or more compounds sufficient to differentiate pluripotent stem cells to chondrocytes, such as TD198946 and/or BMP-2, for a time that is sufficient to differentiate the SBSCs to chondrocytes.

25. A method of differentiating SBSCs to chromaffin cells, comprising contacting the SBSCs with one or more compounds sufficient to differentiate pluripotent stem cells to chromaffin cells, such as BMP-2 and/ or FGF2 for a time that is sufficient to differentiate the SBSCs to chromaffin cells.

26. A method of differentiating SBSCs to cardiomyocytes, comprising contacting the SBSCs with one or more compounds sufficient to differentiate pluripotent stem cells to cardiomyocytes, such as Meglumine, insulin-like growth factor-1, BMP-2, BMP-4, and/or fibroblast growth factors (FGFs) for a time that is sufficient to differentiate the SBSCs to cardiomyocytes.

27. A method of differentiating SBSCs to intestinal organoids, comprising contacting the SBSCs with one or more compounds sufficient to differentiate pluripotent stem cells to intestinal organoids for a time that is sufficient to differentiate the SBSCs to intestinal organoids.

28. The method of alternative 27, wherein the intestinal organoids are positive for CDX2, villin, vimentin, EpCAM, SOX9, cytokeratin, chromogranin A, E-cadherin, MUC2, KLF5, or desmin, or any combination thereof.

29. A method of differentiating SBSCs to liver organoids, comprising contacting the SBSCs with one or more compounds sufficient to differentiate pluripotent stem cells to liver organoids for a time that is sufficient to differentiate the SBSCs to intestinal organoids.

30. A method of differentiating SBSCs to kidney organoids, comprising contacting the SBSCs with one or more compounds sufficient to differentiate pluripotent stem cells to kidney organoids for a time that is sufficient to differentiate the SBSCs to intestinal organoids.

31. The method of alternative 30, wherein the SBSCs are contacted for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any time within a range defined by any two of the aforementioned times.

32. A method of treating or inhibiting chronic kidney disease in a subject in need thereof, comprising administering SBSCs to the subject.

33. A method of treating or inhibiting chronic kidney disease in a subject in need thereof, comprising administering kidney organoids differentiated from SBSCs to the kidney of the subject.

34. The method of alternative 32 or 33, wherein the SBSCs are derived from the subject.

35. The method of alternative 32 or 33, wherein the SBSCs are allogeneic to the subject.

36. The method of any one of alternatives 32-35, wherein the subject is a mammal.

37. The method of any one of alternatives 32-36, wherein the subject is a human, dog, or cat.

38. A method of treating or inhibiting osteoporosis in a subject in need thereof, comprising administering SBSCs to the subject.

39. The method of alternative 38, wherein the SBSCs are administered parenterally.

40. The method of alternatives 38 or 39, further comprising administering parathyroid hormone (PTH) to the subject.

41. The method of any one of alternatives 38-40, wherein the SBSCs are derived from the subject.

42. The method of any one of alternatives 38-41, wherein the SBSCs are allogeneic to the subject.

43. The method of any one of alternatives 38-42, wherein the subject is a mammal.

44. The method of any one of alternatives 38-43, wherein the subject is a human.

45. A method of differentiating SBSCs to hematopoietic cells, comprising contacting the SBSCs with one or more compounds sufficient to differentiate pluripotent stem cells to hematopoietic cells for a time that is sufficient to differentiate the SBSCs to hematopoietic cells.

46. The method of alternative 45, wherein the hematopoietic cells comprise macrophages, erythrocytes, or granulocytes, or any combination thereof.

47. The method of any one of alternatives 23-46, wherein the SBSCs are isolated according to the methods of any one of alternatives 1-19.

48. The method of any one of alternatives 23-47, wherein the SBSCs have been cryopreserved according to the method of alternative 21 or 22.

### BRIEF DESCRIPTION OF THE DRAWINGS

In addition to the features described herein, additional features and variations will be readily apparent from the following descriptions of the drawings and exemplary embodiments. It is to be understood that these drawings depict typical embodiments and are not intended to be limiting in scope.

FIG. 1A depicts an embodiment of the methodology for the isolation of small blood stem cells (SBSCs).

FIG. 1B depicts a representative bright field microscopy image of trypan blue-stained SBSCs in a Neubauer chamber. Black arrows (highlighting white, trypan blue excluded cells) indicate live cells, while gray arrows (highlighting dark, trypan blue-stained cells) indicate dead cells.

FIG. 2 depicts immunofluorescence and light microscopy images of SBSCs. Panel A shows SBSCs stained with KP-1. Panel B shows SBSCs observed in a bright field light microscope. Scale bar represents 5 µm.

FIG. 3A depicts SBSCs stained for embryonic stem cell markers. Panel A shows SBSCs stained with anti-Nanog, anti-SOX-2, anti-CXCR4, and anti-OCT4 antibodies. Panel B shows SBSCs stained with anti-KLF4, anti-cMyc, anti-SSEA-3, and anti-SSEA-4 antibodies. Isotype control staining was examined for each tested antibody and the results were negative. Scale bar represents 5 µm.

FIG. 3B depicts H1 embryonic stem cells stained for embryonic stem cell markers. The cells were stained with anti-Nanog, anti-SOX-2, anti-CXCR4, anti-cMyc, antiOCT4, anti-SSEA-3, anti-SSEA-4, and anti-KLF4 antibodies. Isotype control staining was examined for each tested antibody and the results were negative.

FIG. 4 depicts SBSCs stained for mesenchymal stem cell markers. SBSCs were stained with anti-CD29, anti-PTH1R, anti-CD105, and anti-CD106 antibodies. Isotype control staining was examined for each tested antibody and the results were negative. Scale bar represents 5 µm.

FIG. 5A depicts SBSCs stained for hematopoietic stem cell markers. SBSCs were stained with anti-CD90, anti-CD133, anti-CD45, and anti-CD34 antibodies. Isotype control staining was examined for each tested antibody and the results were negative. Scale bar represents 5 µm.

FIG. 5B depicts SBSCs and white blood cells (WBCs) stained for CD45. Cells were stained with anti-CD45 antibody. Isotype control staining was examined, and the results were negative. Scale bar represents 5 µm.

FIG. 6 depicts SBSCs double-stained for CD45 and embryonic stem cell markers. SBSCs were stained with anti-CD45, anti-CXCR4, anti-Nanog, and anti-SOX-2 antibodies. Isotype control staining was examined for each tested antibody and the results were negative. White arrows indicate simultaneous co-staining of both CD45 and embryonic stem cell markers. Gray arrows indicate strong CD45 staining and weak or absent staining of embryonic stem cell markers. Scale bar represents 5 µm.

FIG. 7A depicts SBSCs isolated from the blood from an equine, canine, camel, and bluefin tuna subject, indicating that these cells are present in animals other than humans.

FIG. 7B depicts SBSCs that have been differentiated into osteoblasts, chondrocytes, chromaffin cells, and beating cardiomyocytes.

FIG. 8 depicts formation and production of intestinal organoids from SBSC over 10 days and during the first passage.

FIG. 9 depicts intestinal organoids that have been stained with DAPI (top row), CDX2, Villin, Vimentin, EpCAM, SOX9, or Desmin (second row), Cytokeratin, Chromogranin A, E-cadherin, MUC2, KLF5 (third row). The bottom row shows a merge of the images in the same column.

FIG. 10 depicts a bright field microscopy image of SBSCs that have been differentiated into kidney specific cells and grown into organoids for 10 days (left) and 11 days (right). White arrows indicate kidney organoids.

FIG. 11 depicts a bright field microscopy image of SBSCs that have been differentiated into kidney specific cells and grown into organoids for 15 days (left). and 11 days (right). White arrows indicate kidney organoids.

FIG. 12 depicts a method for small molecule reversal of aging.

FIG. 13 depicts a method for treatment of osteoporosis with SBSCs.

### DETAILED DESCRIPTION

As disclosed herein, some embodiments relate to populations of small cells isolated from peripheral blood. In some embodiments, the population of small cells isolated from peripheral blood are small blood stem cells (SBSCs). These SBSCs may also be referred to as peripheral blood derived pluripotent stem cells (PBD-PSCs). In some embodiments, SBSCs exhibit pluripotency markers, including but not limited to Nanog, Oct4, SOX-2, CXCR4, cMyc, KLF4, SSEA-3, or SSEA-4, or any combination thereof. In some embodiments, SBSCs are positively stained by Kyoto Probe 1 (KP-1). In some embodiments, SBSCs also exhibit mesenchymal stem cell markers, including but not limited to PTH1R, CD29, CD105, or CD106, or any combination thereof. In some embodiments, SBSCs also exhibit hematopoietic stem cell markers, including but not limited to CD90, CD133, or CD45, or any combination thereof. Also disclosed herein are methods of isolating the populations of small cells isolated from peripheral blood. Also disclosed herein are methods of cryopreserving the populations of small cells isolated from peripheral blood.

Certain aspects of regenerative medicine employs stem cells for the generation of differentiated cells and/or tissues with possible applications in clinical practice. Stem cells are characterized by three main traits: their self-renewal, their differentiation capacity, and their regenerative potential, and can be categorized based on either their differentiation potential or the tissue origin. Depending on their differentiation potential, stem cells can be categorized as totipotent, pluripotent, multipotent, oligopotent, or unipotent. Regarding tissue origin, the two major categories are the embryonic and somatic stem cells. Embryonic stem cells are pluripotent, having the greatest potential for self-renewal and differentiation towards various cell types, whereas somatic stem cells are more confined and varying in characteristics depending on tissue origin. Thus, there has been great interest in embryonic stem cells, as they can be excellent candidates for applications in regenerative medicine, such as cell-based therapies. Nonetheless, there are limitations in using these cells, including their isolation due to ethical reasons, their insufficient population within tissues, their non-autologous nature, and their propensity to form teratomas.

In adults, stem cells can be found in various tissues, such as bone marrow, peripheral blood, adipose tissue, the intestines, and many others. However, in most cases, these cells are characterized as multipotent, giving rise to limited types of different cells. Hematopoietic, mesenchymal, and epithelial stem cells are the three major multipotent stem cells found in adult tissues. Although most multipotent stem cells can be easily isolated and cultured, their use in regenerative medicine is limited due to their restricted differentiation capacity. Therefore, regenerative medicine requires stem cells that are pluripotent, can be easily obtained in large numbers, and do not pose any ethical dilemmas.

The urge to discover natural pluripotent stem cells lead to a breakthrough in the field of regenerative medicine with the discovery of induced pluripotent stem cells (iPSCs). iPSCs are a type of pluripotent stem cell generated in vitro from adult cells by introducing four transcription factors (OCT4, SOX2, MYC, KLF4), also known as the “Yamanaka factors”, into the cell. Expression of the Yamanaka factors activates a signaling cascade that allows reprogramming of skin fibroblasts and other somatic cells into iPSCs. iPSCs can then be preserved for long-term storage, used as part of a coordinated blood banking system, or differentiated into fat, brain, heart, blood, and other specialized cells. The hype with iPSCs is that they would offer an unlimited supply of autologous cells that can be used without risk of immune rejection. The versatility of iPSCs also allows for many other potential uses including disease modeling and elucidating the molecular mechanism of disease, drug screening and discovery, as well as cardiac, liver, and neural toxicity tests. The collective suitability of iPSCs for drug discovery and toxicity testing also potentially allows for iPSCs to serve as human preclinical trials in a test tube. Further, iPSC-based drug discovery has the potential to provide personalized therapies with toxicity prediction quicker, at a lower cost, and with a higher probability of success than conventional drug discovery methods. Additionally, there are many veterinary uses for iPSCs including for human organ generation in domestic animals, cell-based therapies for regenerative medicine, disease modeling in animals, transgenic animal generation, gamete and/or embryo derivation, cellular agriculture and the production of synthetic meats, preservation of biodiversity, biomarker development, drug and toxicity screening, and basic research. However, problems arise with autologous transplantation of iPSCs and their differentiated end-products, including high cost and excessive manufacturing time. This makes them, in most cases, clinically impractical to use. Therefore, currently the solution is to use donor iPSC cell lines to produce allogeneic “off-the-shelf” tissue matching cell products. Although this has reduced the manufacturing time, it is still a relatively expensive approach that clinically demands immunosuppression. Hypothetically, a naturally occurring PSC found in abundance would have low cost, less manufacturing time, and when used autologously, would eliminate the need for immunosuppression. However, the surge in iPSC research has hampered the search for a naturally occurring pluripotent stem cell.

Reyes and Verfaillie isolated a small subpopulation of mesenchymal cells from bone marrow capable of differentiating into mesenchymal cell types including cartilage, bone, fibroblasts, and adipocytes. This subpopulation of cells, termed multipotent adult progenitor cells (MAPCs), can also be differentiated into almost all other mesodermal cell lineages including skeletal, smooth and cardiac myoblasts, and Von-Willebrand factor-positive endothelial cells. Difficulties culturing MAPCs and their limited self-renewal capacity limit their potential uses in regenerative medicine.

The group of Ratajczak identified in adults a population of very small cells bearing markers of pluripotency, including stage-specific antigen (SSEA), nuclear Oct4, Nanog, and Rex1. These cells were termed very small embryonic-like (VSEL) stem cells. VSELs have a size of 5-7 µm in humans and are thought to originate from the germline, as they can give rise to various different types of cells. Due to these properties, VSELs are thought to act as a backup regeneration modality in adult tissues. Nevertheless, the isolation of VSELs from peripheral blood has proven to be a challenge due to their small cell size and limited numbers. VSELs are explored in PCT Publications WO 2007/067280 and WO 2009/059032, each of which is hereby expressly incorporated by reference in its entirety.

In 2007, Virant-Klun et al. isolated from cell suspension of scrapings of ovarian surface epithelium of women, small cells with a diameter of 2-4 µm with similar morphology to VSELs. These cells express embryonic markers of SSEA-3, Oct4, Nanog, and c-Kit, and could differentiate into oocyte-like cells reaching diameters of 95 µm.

In 2008, Matsumoto et al. used ceiling culture techniques to revert mature adipocytes into dedifferentiated fat (DFAT) cells. These cells express hematopoietic stem cell marker CD90 and mesenchymal stem cell markers CD 29 and CD105. Although DFAT cells can differentiate into a variety of different cell types, colonially derived DFAT cells possess heterogenous differentiation potential making it difficult to reliably reproduce experimental results.

As disclosed herein, methods for isolating small cells from peripheral blood employing centrifugation and filtration processing steps have been developed. In some embodiments, these methods may be done using whole blood, and do not involve commonly used density gradient centrifugation and antibody-based cell separation methods. By performing these methods disclosed herein, a new subpopulation of small pluripotent stem cells derived from human peripheral blood but distinct from VSELs were isolated. These small pluripotent stem cells (<5 µm), termed small blood stem cells (SBSCs) express pluripotent embryonic markers including the Yamanaka factors (e.g. Nanog, SOX-2, Oct4, Klf4, c-Myc, SSEA-3), while a fraction of the population also bears hematopoietic (CD45, CD90) and/or mesenchymal markers (CD29, CD105) including parathyroid hormone (PTH) receptors such as parathyroid hormone 1 receptor (PTH1R), suggesting that these cells are a mixed pluripotent population.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

### Definitions

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The “individual”, “patient” or “subject” treated as disclosed herein is, in some embodiments, a mammal. The term “mammal” as used herein includes, but is not limited to, humans, non-human animals, including primates, cattle, sheep, goats, pigs, horses, cats, dogs, rabbits, rodents (e.g., rats or mice), monkeys, etc. The subject can be a subject “in need of” the methods disclosed herein or can be a subject that is experiencing a disease state and/or is anticipated to experience a disease state, and the methods and compositions of the invention are used for therapeutic and/or prophylactic treatment. A subject can be a patient, which refers to a subject presenting to a medical provider for diagnosis or treatment of a disease. A subject can be afflicted with or is susceptible to a disease or disorder but may or may not display symptoms of the disease or disorder.

The terms “effective amount” or “effective dose” as used herein, refers to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein refer to a biological, enzymatic, or therapeutic function.

As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to or at least about 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its ordinary meaning and refers to the performance of a method inside living organisms, usually animals, mammals, including humans, and plants, as opposed to a tissue extract or dead organism.

As used herein, “ex vivo” is given its ordinary meaning and refers to the performance of a method outside a living organism with little alteration of natural conditions.

As used herein, “in vitro” is given its ordinary meaning and refers to the performance of a method outside of biological conditions, e.g., in a petri dish or test tube.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids,

DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein refers to the actual overall amount of the substance, compound, or material relative to the expected overall amount. For example, the yield of the substance, compound, or material may be at least 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein is intended to include any and all solvents, dispersion media, coatings, antibacterial and antifungal agents, isotonic and absorption delaying agents, and the like, compatible with administration to humans or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans. The term diluent, excipient, and/or “carrier” can refer to a vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, or ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, saltforming counterions such as sodium, and polyethylene glycol (PEG). The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals.

Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, human serum, or fetal bovine serum [FBS]) to enhance post-thawing survivability of the cells. In these compositions, DMSO may be found at a concentration of 2%, 3%, 4%, 5%, 6%, 7%, 8%, 9%, 10%, 11%, 12%, 13%, 14%, 15%, 16%, 17%, 18%, 19% or 20%, or any percentage within a range defined by any two of the aforementioned numbers. In these compositions, human serum may be found at a concentration of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%, or any percentage within a range defined by any two of the aforementioned numbers. Other cryopreservation media are known in the art, including but not limited to mFreSR and Bambanker medium.

Additional excipients with desirable properties include but are not limited to preservatives, adjuvants, stabilizers, solvents, buffers, diluents, chelating agents, antioxidants, alcohols, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, gelatin, esters, ethers, 2-phenoxyethanol, or vitamins, or any combination thereof. Some excipients may be in residual amounts or contaminants from the process of manufacturing, including but not limited to serum, albumin, ovalbumin, antibiotics, inactivating agents, gelatin, cell debris, nucleic acids, peptides, amino acids, or growth medium components or any combination thereof. The amount of the excipient may be found in composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di, and triethanolamine; amino acids, including glycine, arginine and lysine; guanidine; Nmethylglucosamine; N-methylglucamine; L-glutamine; N-methylpiperazine; morpholine; ethylenediamine; N-benzylphenethylamine; or trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” refers to a compound, particle, solid, semi-solid, liquid, or diluent that facilitates the passage, delivery and/or incorporation of a compound to cells, tissues and/or bodily organs.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The pharmaceutical compound can also be administered in a local rather than systemic manner, for example, via injection of the compound directly into an organ, tissue, cancer, tumor, infected area, or otherwise diseased region.

### Small Blood Stem Cells (SBSCs)

SBSCs were isolated from human peripheral blood using a unique protocol based on centrifugation and filtration of blood without the use of density gradients or antibody-based cell separation methods. SBSCs isolated according to this protocol express known pluripotency markers (KP-1, Nanog, Oct4, SOX-2, CXCR4, cMyc, KLF4, SSEA-3, and SSEA-4). A population of SBSCs also express mesenchymal (CD29, CD105, CD106) and/or hematopoietic (CD90, CD133, CD45) markers, demonstrating that the isolated cells are a mixed pluripotent population that includes a fraction with mesenchymal and hematopoietic characteristics, resembling previous observations of heterogenicity of small stem cells in umbilical cord blood.

This pluripotent subpopulation of small peripheral blood cells uniquely stained with KP-1. KP-1 has also been used as a component of synthetic hybrid molecules that can target, attach to, and isolate pluripotent stem cells from cell mixtures, such as to avoid tumorigenesis. Moreover, the other embryonic cell markers identified are core elements of pluripotency. SSEA-3 is the selective marker to distinguish multilineage-differentiating stress-enduring (MUSE) cells, a pluripotent subpopulation of cells found among mesenchymal stem cells and fibroblasts. SSEA-4 characterizes primate embryonic stem cells and is used in the characterization and establishment of known embryonic stem cell lines derived from human blastocytes (H1, H9). Nanog is the “master gene” of embryonic stem cell pluripotency and was shown to work with other key pluripotency factors such as Oct4 and SOX-2 to control the set of target genes important for embryonic cell pluripotency. CXCR4 is a chemokine receptor expressed in leukocytes, epithelial cells, and embryonic stem cells. The Yamanaka factors (Oct4, KLF4, SOX-2, and cMyc) have been established as being important in the process of creating induced pluripotent stem cells. A population of SBSCs isolated through the processes disclosed herein were found to be positive for these markers. This evidence demonstrates that the SBSCs identified herein have a pluripotent character.

Populations of SBSCs were also found to be positive for CD29 and CD105, which are widely studied markers for mesenchymal stem cells, suggesting that they may have the capability towards mesenchymal lineage differentiation. On the other hand, CD106, which was also found in a small portion of SBSCs, is a marker primarily expressed in endothelial cells and secondarily in some mesenchymal stem cells, suggesting that a subpopulation of SBSCs may have the capacity for endothelial cell differentiation.

Furthermore, the cells were found to stain for membrane-bound PTH1 receptor (PTH1R). It was previously proposed that VSELs may have an important role in bone metabolism and hence, may express parathyroid hormone (PTH) receptors. PTH receptors have been shown to be expressed by mesenchymal stem cells and are thought to be important for driving the anabolic effect of bone that is observed with intermittent PTH and parathyroid hormone-related protein (PTHrP) agonists. This characteristic of SBSCs, which are more primitive in origin than mesenchymal stem cells, due to their size, large quantity, and pluripotent nature, suggests that these cells may have an important role in bone metabolism.

Finally, the hematopoietic marker CD90, a surface marker of different types of cells, including T cells and mesenchymal stem cells, were found to be expressed in SBSCs, suggesting again that these cells may have a mixed phenotype. Regarding CD133, although it was initially described as a stem cell marker of hematopoietic origin, it was later used for the identification of VSELs. In contrast to previous studies of VSEL characterization, the SBSCs isolated through the methods disclosed herein were found to be CD45 positive but CD34 negative.

Among the many advantages pluripotent stem cells provides: three-dimensional spatial organization; genetically stable; preservation of genetic and epigenetic signature of derived tissue; long-term culture; biobanks; safe for transplantation; unlimited source of patient-derived cells; non-invasive derivation from a variety of cells (e.g., skin/fibroblasts/blood cells); recapitulate different aspects of liver development; disease modelling; high-throughput drug screening; personalized medicine; and gene therapy; only PBD-PSCs provide every advantage.

Disclosed herein are methods of isolating small blood stem cells (SBSCs) from peripheral blood. In some embodiments, the methods comprise centrifuging the peripheral blood to isolate plasma from the peripheral blood, centrifuging the plasma to isolate a cell pellet comprising the SBSCs, resuspending the cell pellet, and filtering the resuspended cell pellet through a filter having a pore size to exclude the cells that are larger than the pore size, thereby isolating the SBSCs. In some embodiments, the peripheral blood is from a subject. In some embodiments, the peripheral blood is from a human subject. In some embodiments, the peripheral blood is from a healthy subject. In some embodiments, the peripheral blood is centrifuged at a speed that is, is at least, or is not more than, 200×g, 250×g, 300×g, 350×g, 400×g, 450×g, 500×g, 550×g, 600×g, 650×g, 700×g, 750×g, or 800×g, or about 200×g, about 250×g, about 300×g, about 350×g, about 400×g, about 450×g, about 500×g, about 550×g, about 600×g, about 650×g, about 700×g, about 750×g, or about 800×g, or any speed within a range defined by any two of the aforementioned speeds. In some embodiments, the plasma is centrifuged at a speed that is, is at least, or is not more than, 1000×g, 1050×g, 1100×g, 1150×g, 1200×g, 1250×g, 1300×g, 1350×g, or 1400×g, or about 1000×g, about 1050×g, about 1100×g, about 1150×g, about 1200×g, about 1250×g, about 1300×g, about 1350×g, or about 1400×g, or any speed within a range defined by any two of the aforementioned speeds. In some embodiments, the peripheral blood is centrifuged with a density gradient. In some embodiments, the peripheral blood is centrifuged without a density gradient. In some embodiments, the plasma is centrifuged with a density gradient. In some embodiments, the peripheral blood is centrifuged without a density gradient. In some embodiments, the pore size of the filter is 5 µm. In some embodiments, the cells that are larger than 5 µm are excluded by the filtering step. In some embodiments, the pore size of the filter is larger than 5 µm. In some embodiments, the pore size of the filter is, is at least, or is not more than, 1 µm, 2 µm, 3 µm, 4 µm, 5 µm, 6 µm, 7 µm, 8 µm, 9 µm, 10 µm, 11 µm, 12 µm, 13 µm, 14 µm, 15 µm, 16 µm, 17 µm, 18 µm, 19 µm, or 20 µm, or about 1 µm, about 2 µm, about 3 µm, about 4 µm, about 5 µm, about 6 µm, about 7 µm, about 8 µm, about 9 µm, about 10 µm, about 11 µm, about 12 µm, about 13 µm, about 14 µm, about 15 µm, about 16 µm, about 17 µm, about 18 µm, about 19 µm, or about 20 µm, or any size within a range defined by any two of the aforementioned sizes. In some embodiments, the cell pellet is resuspending in an isotonic solution. In some embodiments, the isotonic solution is growth medium, 0.9% saline, 5% dextrose solution, Ringer’s lactate solution, or Ringer’s acetate solution, or any combination thereof. In some embodiments, the peripheral blood is mixed with an anticoagulant prior to centrifuging. In some embodiments, the anticoagulant is sodium citrate. In some embodiments, the anticoagulant is a 3%-4% solution of sodium citrate.

In some embodiments, the SBSCs isolated by any one of the methods described herein are disclosed. In some embodiments, the SBSCs isolated by any of the methods described herein comprise populations of cells comprising pluripotency markers, mesenchymal stem cell markers, or hematopoietic stem cell markers, or any combination thereof. In some embodiments, the pluripotency markers comprise one or more (e.g. at least 1, 2, 3, 4) of Nanog, Oct4, SOX-2, CXCR4, cMyc, KFL4, SSEA-3, or SSEA-4, or any combination thereof. In some embodiments, the mesenchymal stem cell markers comprise one or more (e.g. at least 1, 2, 3, 4) of CD29, PTH1R, CD105, or CD106, or any combination thereof. In some embodiments, the hematopoietic stem cell markers comprise one or more (e.g. at least 1, 2, 3) of CD90, CD133, or CD45, or any combination thereof. In some embodiments, the SBSCs stain with Kyoto Probe 1. In some embodiments, the SBSCs isolated by any one of the methods described herein are, are about, are at least, are at least about, are not more than, or are not more than about, 0.5 µm, 1 µm, 2 µm, 3 µm, 4 µm, 5 µm, 6 µm, 7 µm, 8 µm, 9 µm, or 10 µm in diameter, or any diameter within a range defined by any two of the aforementioned diameters.

Also disclosed herein are methods of cryopreserving SBSCs. In some embodiments, the SBSCs are isolated by any one of the methods described herein. In some embodiments, the methods of cryopreserving SBSCs comprise resuspending the SBSCs in a cryopreservation medium, freezing the SBSCs at -80° C., and transferring the frozen SBSCs to -150° C. In some embodiments, the SBSCs are resuspended in the cryopreservation medium after the filtering step of the methods of isolation described herein. In some embodiments, the cryopreservation medium is a 10:1 ratio of human serum:DMSO, mFreSR medium, or Bambanker medium, or any combination thereof. In some embodiments, the SBSCs are thawed after cryopreservation. In some embodiments, the SBSCs are thawed by removing from the 150° C. storage, incubating at room temperature, and incubating at 37° C., thereby thawing the SBSCs. The thawed SBSCs are resuspended in growth medium and the cryopreservation medium is removed, such as by centrifugation.

### Methods of Use

In some embodiments, the SBSCs isolated by any of the methods disclosed herein may be used to differentiate into downstream cell types, for example, for use in production of organoids, transplantation, or treatment or inhibition of a disease.

In some embodiments are methods of differentiating SBSCs to osteoblasts. In some embodiments, the methods comprise contacting the SBSCs with one or more compounds sufficient to differentiate pluripotent stem cells to osteoblasts, such as purmorphamine, a 2,6,9-trisubstituted purine compound, ascorbic acid, P-glycerophosphate, dexamethasone or retinoic acid, or any combination thereof, or by co-culture with fetal murine osteoblasts for a time that is sufficient to differentiate the SBSCs to osteoblasts.

In some embodiments are methods of differentiating SBSCs to chondrocytes. In some embodiments, the methods comprise contacting the SBSCs with one or more compounds sufficient to differentiate pluripotent stem cells to chondrocytes, such as TD198946 or BMP-2, or both, for a time that is sufficient to differentiate the SBSCs to chondrocytes.

In some embodiments are methods of differentiating SBSCs to chromaffin cells. In some embodiments, the methods comprise contacting the SBSCs with one or more compounds sufficient to differentiate pluripotent stem cells to chromaffin cells, such as BMP-2 or FGF2, or both, for a time that is sufficient to differentiate the SBSCs to chromaffin cells.

In some embodiments are methods of differentiating SBSCs to cardiomyocytes. In some embodiments, the methods comprise contacting the SBSCs with one or more compounds sufficient to differentiate pluripotent stem cells to cardiomyocytes, such as meglumine, insulin-like growth factor-1, BMP-2, BMP-4, or fibroblast growth factors (FGFs), or any combination thereof, for a time that is sufficient to differentiate the SBSCs to cardiomyocytes.

In some embodiments are methods of differentiating SBSCs to intestinal organoids. In some embodiments, the methods comprise contacting the SBSCs with one or more compounds sufficient to differentiate pluripotent stem cells to intestinal organoids for a time sufficient to differentiate the SBSCs to intestinal organoids. In some embodiments, the intestinal organoids are positive for CDX2, villin, vimentin, EpCAM, SOX9, cytokeratin, chromogranin A, E-cadherin, MUC2, KLF5, or desmin, or any combination thereof.

In some embodiments are methods of differentiating SBSCs to liver organoids. In some embodiments, the methods comprise contacting the SBSCs with one or more compounds sufficient to differentiate pluripotent stem cells to liver organoids for a time that is sufficient to differentiate the SBSCs to intestinal organoids.

In some embodiments are methods of differentiating SBSCs to kidney organoids. In some embodiments, the methods comprise contacting the SBSCs with one or more compounds sufficient to differentiate pluripotent stem cells to kidney organoids for a time that is sufficient to differentiate the SBSCs to intestinal organoids.

In some embodiments, are methods of differentiating SBSCs to hematopoietic cells. In some embodiments, the methods comprise contacting the SBSCs with one or more compounds sufficient to differentiate pluripotent stem cells to hematopoietic cells for a time that is sufficient to differentiate the SBSCs to hematopoietic cells. In some embodiments, the hematopoietic cells comprise macrophages, erythrocytes, or granulocytes, or any combination thereof.

As applied to any one of the methods for differentiation, in some embodiments, the SBSCs are contacted for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any time within a range defined by any two of the aforementioned times.

Also disclosed herein are methods of treating or inhibiting chronic kidney disease in a subject in need thereof. In some embodiments, the methods comprise administering SBSCs to the subject. In some embodiments, the SBSCs are derived from the subject. In some embodiments, the SBSCs are allogeneic to the subject. In some embodiments, the subject is a mammal. In some embodiments, the subject is a human, dog, or cat.

Also disclosed herein are methods of treating or inhibiting chronic kidney disease in a subject in need thereof. In some embodiments, the methods comprise administering kidney organoids differentiated from SBSCs to the kidney of the subject. In some embodiments, the SBSCs are derived from the subject. In some embodiments, the SBSCs are allogeneic to the subject. In some embodiments, the subject is a mammal. In some embodiments, the subject is a human, dog, or cat.

Also disclosed herein are methods of treating or inhibiting osteoporosis in a subject in need thereof. In some embodiments, the methods comprise administering SBSCs to the subject. In some embodiments, the SBSCs are administered parenterally. In some embodiments, the methods further comprise administering parathyroid hormone (PTH) to the subject. In some embodiments, the SBSCs are derived from the subject. In some embodiments, the SBSCs are allogeneic to the subject. In some embodiments, the subject is a mammal. In some embodiments, the subject is a human.

As applied to any of the methods of differentiation and/or treatment or inhibition disclosed herein, in some embodiments, the SBSCs are isolated by one or more of the isolation methods disclosed herein.

As applied to any of the methods of differentiation and/or treatment or inhibition disclosed herein, in some embodiments, the SBSCs have been cryopreserved according to any one or more of the cryopreservation methods disclosed herein.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the invention, as it is described herein and in the claims.

### Example 1. Materials and Methods

Healthy volunteers: Peripheral blood was collected from healthy human volunteers, who were first informed and agreed to participate. They were of all sexes and their age varied from 20-30 years old.

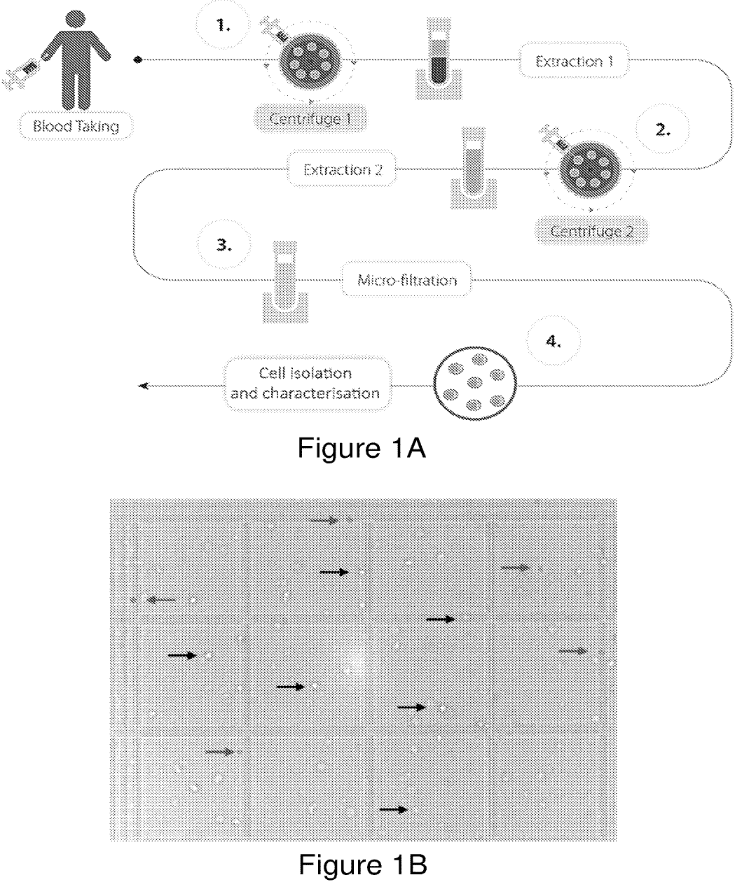

Isolation of SBSCs: FIG. 1A shows an example of the process for isolating SBSCs. Briefly, peripheral blood was collected in tubes containing 3-4% sodium citrate and the tubes were then centrifuged at 600×g for 10 minutes in order to separate red and white blood cells from the plasma layer. The plasma layer was then carefully transferred into a new centrifuge tube and centrifuged again at 1200×g for another 10 minutes. Supernatant plasma was then discarded and the pellet containing the SBSCs was resuspended in sterile saline. Finally, the suspension was filtered through a 5 µm syringe driven filter and SBSCs were collected for further studies.

SBSC counting: SBSCs were isolated from healthy young volunteers and appropriate dilutions with sterile saline were made. SBSCs were then stained with 0.4% trypan blue solution to exclude any possible dead cells and debris, and 10 µL of sample was loaded on each loading groove of a hemocytometer. Using a 40× objective, the five RBC squares of the counting chamber were identified and the live population of SBSCs were counted (FIG. 1B). The final number of live SBSCs was calculated using the formula: SBSCs per µL = counted SBSCs/(counted surface × chamber depth × dilution factor). Finally, the number of isolated SBSCs was translated into the number of SBSCs found in the collected plasma from peripheral blood per sample.

Kyoto Probe 1 Staining: SBSCs were stained using Kyoto Probe 1 (KP-1; Goryo Chemical) according to the manufacturer’s instructions. Briefly, SBSCs were centrifuged at 1200×g for 10 minutes and the pellet was then reconstituted in phosphate buffered saline (PBS). Next, 10 µg of KP-1 was dissolved in DMSO to prepare a 5 mM stock solution and then diluted again in PBS to prepare a 2 µM working cell stain solution. Stain solution was then added to the cells, incubated for 3 hours, and culture slides were studied with a fluorescence microscope.

Immunofluorescence: SBSCs were characterized using immunofluorescence. Cells were centrifuged at 1200×g for 10 minutes and the pellet was then reconstituted and fixed in ice-cold 4% paraformaldehyde (PFA) for 20 minutes at 4° C. After fixation, cells were washed in PBS and then permeabilized with 0.05% Triton X-100 in 1× PBS for 10 minutes. Nonspecific staining was subsequently blocked with a 60-minute incubation with 5% bovine serum albumin. The slides were then incubated at room temperature for 1 hour with antibodies against isotype controls or targets of interest at a 1:50 dilution. The targets of interest included embryonic markers (Nanog, Oct4, CXCR4, SOX2, KLF4, cMyc, SSEA-3, SSEA-4), mesenchymal markers (CD29, PTH1R, CD105, CD106), and hematopoietic markers (CD45, CD34, CD90, CD133). Next, nuclei were stained with DAPI and culture slides were studied with a fluorescence microscope.

Culture of Human Embryonic Stem Cell Line H1: The human embryonic stem cell line H1 (Wicell International Stem Cell Bank) was cultured and served as a positive control in antibody verification of embryonic stem cell markers. H1 stem cells were cultured in 6-well plates coated with Matrigel (BD Biosciences), fed daily with mTeSR1 (StemCell Technologies) and observed under microscope for any spontaneous differentiation. When confluence reached around 90%, H1 stem cells were passaged, seeded in Matrigel-coated chamber slides (ibidi GmbH) and later stained for the detection of embryonic stem cell markers using immunofluorescence.

Isolation of White Blood Cells (WBCs): Peripheral blood was collected in tubes containing 3-4% sodium citrate. Equal parts of whole blood and sterile saline were mixed and then applied over Biocoll separating solution (Sigma). Samples were then centrifuged at 1200×g for 20 minutes and an enriched layer of WBC was later formed and retrieved using a Pasteur pipette. WBC were then washed twice using sterile saline and used for further studies.

### Example 2. Small Cells Isolated From Peripheral Blood Are Pluripotent

SBSCs were isolated from peripheral blood and counted using Neubauer chambers as described in Example 1. After staining with trypan blue, a large number of live SBSCs was observed (FIG. 1B). The mean of counted live populations of SBSCs/µL was 74.133 cells/µL (SEM: 6.919) which corresponds to a mean of 46,912,356 cells/mL of plasma (SEM: 15,920,184).

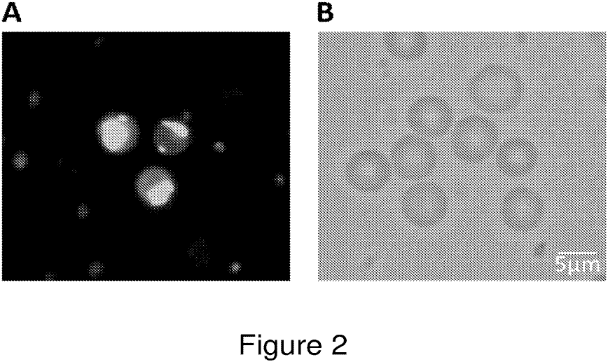

SBSCs isolated from human peripheral blood were stained with Kyoto Probe 1 (KP-1) for the detection of pluripotency. Pluripotent cells stained with KP-1 fluoresce in the green spectrum due to their lack of ABC transporters, which normally eliminate this probe in somatic or differentiated cells. KP-1 staining resulted in a positive stained subpopulation of peripheral blood small cells, suggesting that these cells are pluripotent stem cells (FIG. 2).

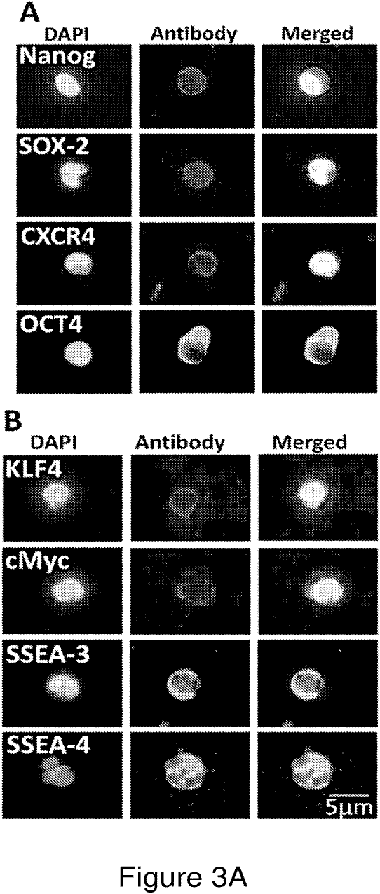

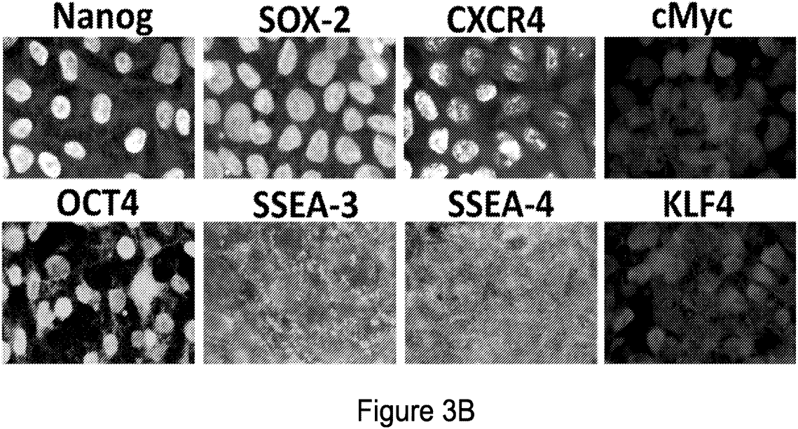

To confirm that these SBSCs have the ability to be pluripotent, they were stained with antibodies against pluripotent markers Nanog, CXCR4, SSEA-3 and SSEA-4, and the Yamanaka pluripotency factors Oct4, SOX-2, cMyc and KLF4, known to be expressed in embryonic stem cells. SBSCs expressed all investigated pluripotent markers (FIG. 3A), which were also present in the positive control H1 embryonic stem cell line (FIG. 3B).

### Example 3. SBSCs Express Markers of Mesenchymal Origin

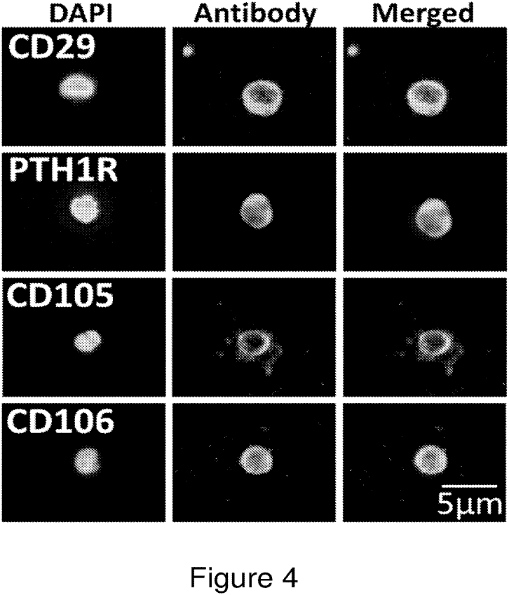

The identity of SBSCs were further investigated by staining against known markers (CD29, PTH1R, CD105, CD106) found in mesenchymal stem cells. A portion of SBSCs expressed these markers (FIG. 4); approximately 80%, 90%, 10%, and 10% were found to be positive for CD29, PTH1R, CD105, and CD106, respectively, suggesting that these stem cells may be able to differentiate towards mesenchyme lineages.

### Example 4. SBSCs Comprise Both Embryonic and Hematopoietic Stem Cells

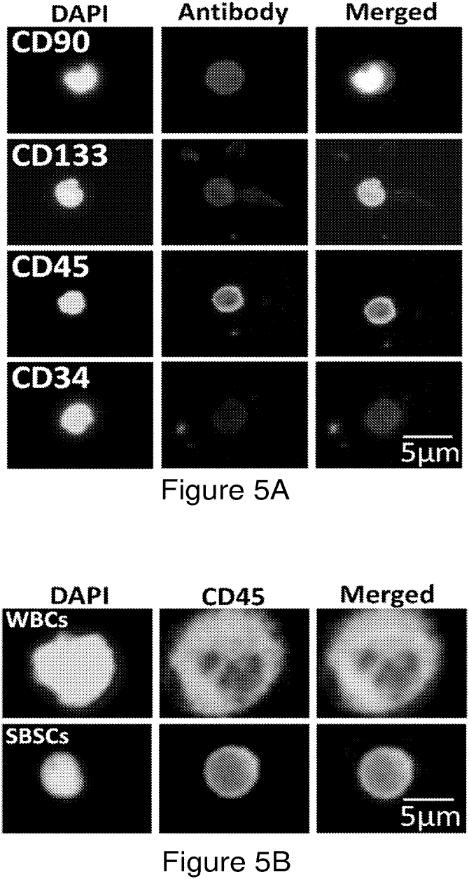

SBSCs were examined for characteristic factors of hematopoietic origin (CD90, CD133, CD45, CD34). Indeed, SBSCs were found to be positive for CD90, CD133, and CD45, but not for CD34 (FIG. 5A). To confirm this, SBSCs and WBCs were both stained with CD45 antibody to exclude any possible false positive artifacts. Both SBSCs and WBCs expressed CD45 (FIG. 5B).

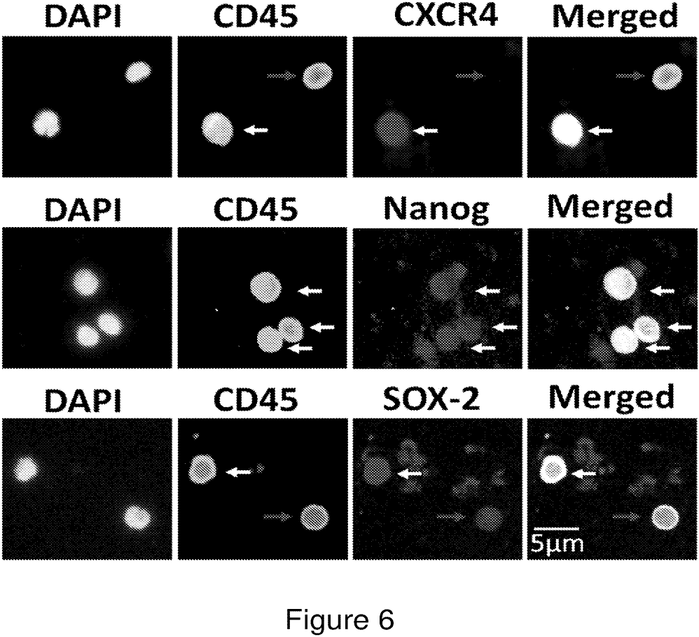

SBSCs were simultaneously double-stained with antibodies against CD45 and the embryonic stem cell markers CXCR4, Nanog, and SOX-2. A mixed population of SBSCs were observed, with some expressing both CD45 and embryonic stem cell markers (FIG. 6, white arrows) with others expressing only CD45 and with weak or no expression of embryonic stem cell markers (FIG. 6, gray arrows). These results provide evidence that SBSCs are a mixed population of embryonic stem cell-like cells that express certain hematopoietic markers.

### Example 5. Cryopreservation of SBSCs

After isolation of SBSCs from peripheral blood, cells can be cryopreserved for later use. To the prepared SBSCs after filtration, the SBSCs are centrifuged and the SBSC pellet is resuspended in 1 mL of a 10:1 human serum:DMSO solution, mFreSR (Stem Cell Technologies), or Bambanker (Nippon Genetics) freezing media is added per centrifugation vial. The cells are resuspended in the freezing media and transferred to a cryovial. Cells are cryopreserved at -80° C. for at least 24 hours in an isopropanol-filled freezing chamber. After 24 hours, cryovials are transferred for storage at -80° C. or -150° C.

To thaw the cells, cryovials are removed from storage and incubated at room temperature for 1 minute. If needed, the vial lid can be loosened to reduce pressure differential. After closing the lid, the cryovial is incubated in a 37° C. water bath until thawed. Cells are resuspended gently with a pipette and transferred into a tube containing 10 mL of growth media or PBS. The cells are centrifuged at 1200×g for 10 minutes. The supernatant is discarded, and cells are resuspended in 2 mL of fresh growth media or PBS.

### Example 6. SBSCs Can Be Found in Other Animals

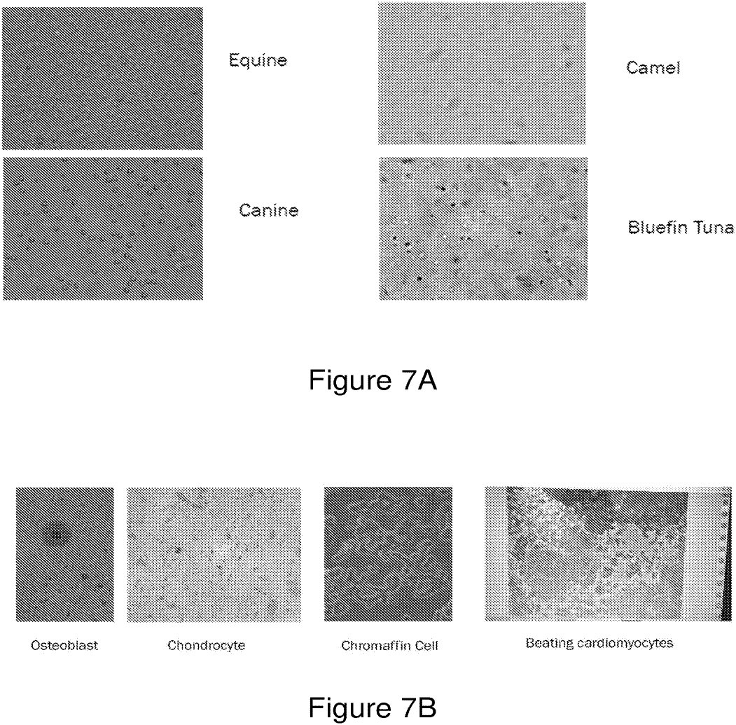

SBSCs have also been isolated from the blood from equine, canine, camel, and bluefin tuna subjects (FIG. 7A).

### Example 7. Differentiation of SBSCs

After isolation of SBSCs from peripheral blood, cells can be differentiated into multiple different cell types including osteoblasts, chondrocytes, chromaffin cells, and beating cardiomyocytes (FIG. 7B).

### Example 8. Use of SBSC Derived Chromaffin Cells for Treatment of Chronic Pain

Following differentiation of SBSCs into chromaffin cells, the chromaffin cells are washed and suspended in a chronic pain subject’s cerebral spinal fluid in preparation for transplantation into the subject’s subarachnoid space.

### Example 9. Use of SBSC Derived Organoids Cells for Treatment as a Cure or Treatment for Disease in Subjects Awaiting an Organ Transplantation

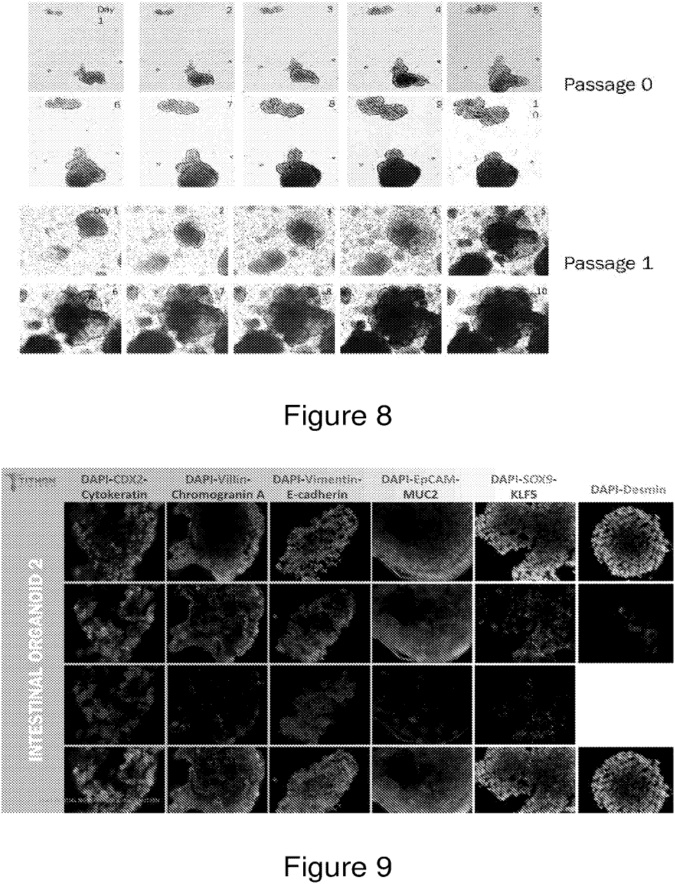

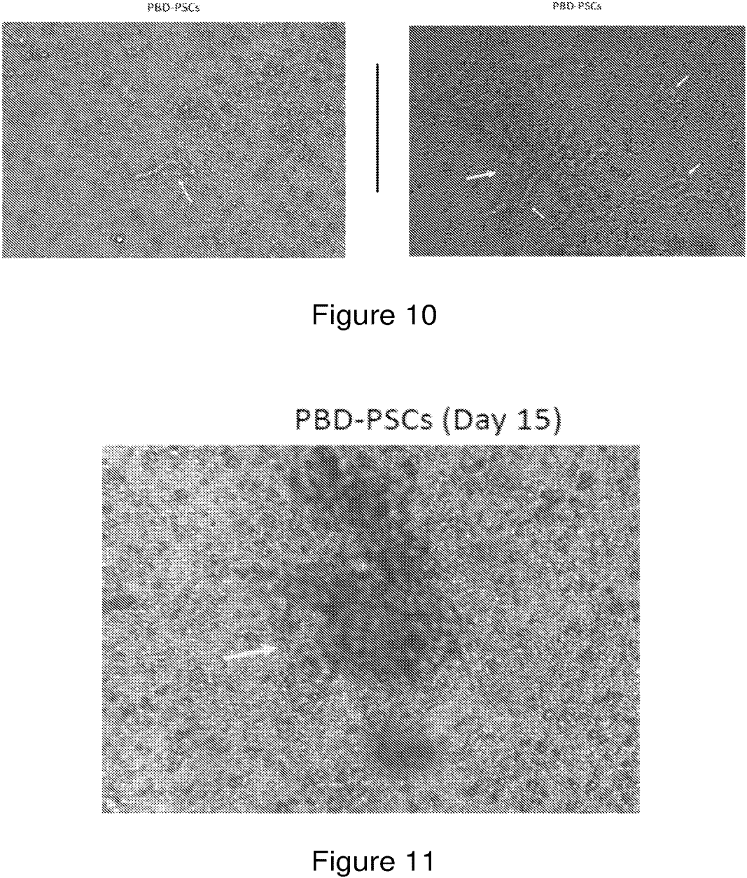

Following isolation of SBSCs from peripheral blood, the SBSCs were differentiated into tissue specific organoids such as intestinal (FIG. 8 and FIG. 9), kidney (FIG. 10 and FIG. 11), and liver. Kidney and liver organoid tissues are then transplanted back into subjects with failing kidneys and/or livers who are awaiting an organ transplant.

### Example 10. Use of SBSC Derived Kidney Organoids and Tubuloids for Studying and Treating Chronic Kidney Disease in Human Subjects

Following isolation of SBSCs from peripheral blood, the SBSC are differentiated into kidney organoids and tubuloids. The differentiated kidney tissues are then used to study kidney development, renewal, and regeneration; modeling of chronic kidney disease; creation and maintenance of kidney specific biobanks; a personalized determination of drug efficacy and toxicity screening; and regenerative nephrology.

### Example 11. Use of SBSCs, or Kidney Organoids or Tubuloids Derived Thereof, for Studying And Treating Nephron Damage Associated With Chronic Kidney Disease in Dogs and Cats

A 5 year old female (spayed) French Bulldog canine subject was diagnosed with congenital renal dysplasia. The subject was treated 6 times in 2015 with her own adipose-derived mesenchymal stem cells combined with platelet-rich plasma (PRP). She was kept in Stage 1 failure for approximately 4 months before she would require a retreatment, with her values heading to Stage 2 renal disease.

In 2016, the subject was compassionately treated with SBSCs placed peri-renally in the retroperitoneal space. Following this treatment, the subject remained bright and alert throughout the entire year, without relapse. Previously, she relapsed every 3-4 months following mesenchymal stem cell treatment. Table 1 depicts exemplary measurements of kidney function of the subject. The SBSCs were administered to the subject on Jan. 26, 2016. The subject exhibited a decline at Mar. 10, 2016, but recovered by May 29, 2016.

TABLE 1

Kidney function of a canine subject treated with SBSCs

| | Blood urea nitrogen (BUN) (Normal range: 6-26 mg/dL) | Creatinine (Normal range: 0.5 - 1.6 mg/dL) | Symmetric dimethylarginine (SDMA) (Normal range: less than 14 µg/dL) | Urinalysis |
|---|---|---|---|---|
| Jan. 26, 2016 (time of SBSC intervention) | 19 | 1.7 | Not done | Within normal limits (WNL) |
| Mar. 10, 2016 | 12 | 0.3 | Not done | WNL |
| May 29, 2016 | 20 | 1.6 | 13 | WNL |
| Aug. 9, 2016 | 19 | 1.8 | 17 | WNL |
| Sep. 22, 2016 | 19 | 1.6 | 20 | WNL |

### Example 12. Small Molecule Reprogramming of Aging

Aging is characterized by a gradual loss of function occurring at the molecular, cellular, tissue and organismal levels. At the chromatin level, aging is associated with the progressive accumulation of epigenetic errors that eventually lead to aberrant gene regulation, stem cell exhaustion, senescence, and deregulated cell / tissue homeostasis. The technology of nuclear reprogramming to pluripotency, through over-expression of the Yamanaka factors, can revert both the age and identity of any cell to that of an embryonic cell by driving epigenetic reprogramming. Transient transgenic reprogramming can ameliorate age-associated hallmarks and extend lifespan in progeroid mice.

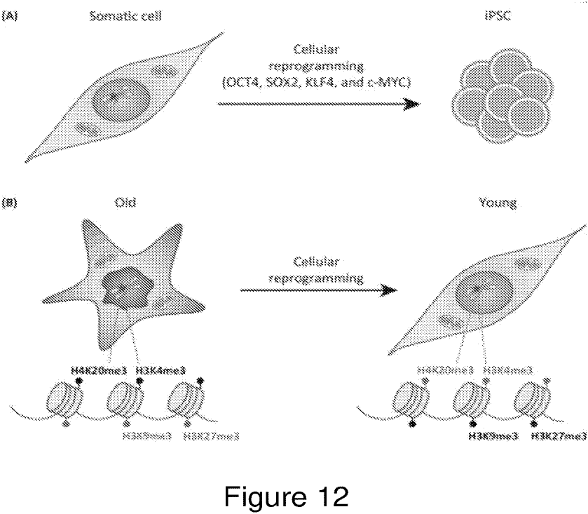

These methods generally involve the insertion of genetic material into old cells with an abnormal nuclear envelope, undergoing senescence, with mitochondrial dysfunction, or dysregulation of epigenetic marks, for expression of the Yamanaka factors. However, this reprogramming can be accomplished by using a non-toxic small molecule cocktail, in vitro, without the need for genetic manipulation. This small molecule reprogramming of aging (SMRA) paves the way to a novel, translatable strategy for ex-vivo cell rejuvenation treatment. SMRA additionally allows for in vivo tissue rejuvenation therapies to reverse the physiological manifestations of aging and the risk for the development of age-related-diseases (FIG. 12).

### Example 13. Use of PTH1R Positive SBSCs for Treatment of Osteoporosis

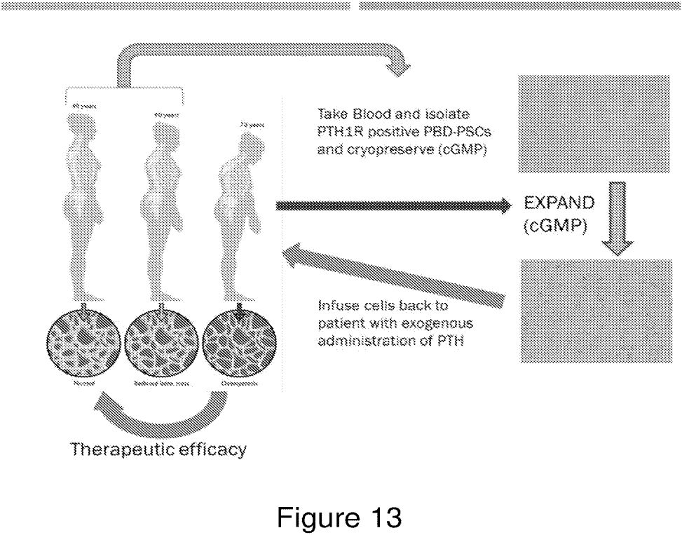

The global osteoporosis drug market is expected to reach USD 16.3 billion by 2025. An upsurge rise in the unhealthy lifestyle adoption has resulted in aggravation and increase in the prevalence of osteoporosis which is presumed to propel the osteoporosis drug market during the forecast period. To treat osteoporosis, autologous, PTH1R positive SBSCs are isolated from the subject’s peripheral blood and cryopreserved. The PTH1R positive SBSC culture is then expanded and infused back into the subject along with exogenous administration of PTH, resulting in the reversal of osteoporosis (FIG. 13).

### Example 14. Treatment of Muscular Dystrophy

Diseased cells from subjects with muscular dystrophy are isolated from the subject and cultured to expand the cell population. CRISPR is then used to genetically reprogram the diseased cells. The gene corrected cells are then injected back into the subject where they can form healthy muscle tissue.

The invention is generally disclosed herein using affirmative language to describe the numerous embodiments. The invention also includes embodiments in which subject matter is excluded, in full or in part, such as substances or materials, method steps and conditions, protocols, or procedures.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” [0143] In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Ahlström M, Pekkinen M, & Lamberg-Allardt C (2009). Dexamethasone downregulates the expression of parathyroid hormone-related protein (PTHrP) in mesenchymal stem cells. Steroids 74: 277-282.

Alvarez-Gonzalez C, Duggleby R, Vagaska B, Querol S, Gomez SG, Ferretti P, et al. (2013). Cord blood Lin(-)CD45(-) embryonic-like stem cells are a heterogeneous population that lack self-renewal capacity. PloS one 8: e67968.

Bamias G, Filidou E, Goukos D, Valatas V, Arvanitidis K, Panagopoulou M, et al. (2017). Crohn’s disease-associated mucosal factors regulate the expression of TNF-like cytokine 1A and its receptors in primary subepithelial intestinal myofibroblasts and intestinal epithelial cells. Translational research : the journal of laboratory and clinical medicine 180: 118-130.e112.

Brown C, McKee C, Bakshi S, Walker K, Hakman E, Halassy S, et al. (2019). Mesenchymal stem cells: Cell therapy and regeneration potential. J Tissue Eng Regen Med 13: 1738-1755.

Chang YJ, Tien KE, Wen CH, Hsieh TB, & Hwang SM (2014). Recovery of CD45(-)/Lin(-)/SSEA-4(+) very small embryonic-like stem cells by cord blood bank standard operating procedures. Cytotherapy 16: 560-565.

De Luca M, Aiuti A, Cossu G, Parmar M, Pellegrini G, & Robey PG (2019). Advances in stem cell research and therapeutic development. Nature cell biology 21: 801-811.

Filidou E, Valatas V, Drygiannakis I, Arvanitidis K, Vradelis S, Kouklakis G, et al. (2018). Cytokine receptor profiling in human colonic subepithelial myofibroblasts: a differential effect of Th polarization associated cytokines in intestinal fibrosis. InflammBowel Dis.

Galkowski D, Ratajczak MZ, Kocki J, & Darzynkiewicz Z (2017). Of Cytometry, Stem Cells and Fountain of Youth. Stem Cell Rev Rep 13: 465-481.

Halasa M, Baskiewicz-Masiuk M, Dabkowska E, & Machalinski B (2008). An efficient two-step method to purify very small embryonic-like (VSEL) stem cells from umbilical cord blood (UCB). Folia Histochem Cytobiol 46: 239-243.

Halfon S, Abramov N, Grinblat B, & Ginis I (2011). Markers distinguishing mesenchymal stem cells from fibroblasts are downregulated with passaging. Stem cells and development 20: 53-66.

Hirata N, Nakagawa M, Fujibayashi Y, Yamauchi K, Murata A, Minami I, et al. (2014). A chemical probe that labels human pluripotent stem cells. Cell Rep 6: 1165-1174. [0158] Horn PA, Tesch H, Staib P, Kube D, Diehl V, & Voliotis D (1999). Expression of AC133, a novel hematopoietic precursor antigen, on acute myeloid leukemia cells. Blood 93: 1435-1437.

Ise H, Matsunaga K, Shinohara M, & Sakai Y (2019). Improved Isolation of Mesenchymal Stem Cells Based on Interactions between N-Acetylglucosamine-Bearing Polymers and Cell-Surface Vimentin. Stem Cells Int 2019: 4341286.

Jordan NJ, Kolios G, Abbot SE, Sinai MA, Thompson DA, Petraki K, et al. (1999). Expression of functional CXCR4 chemokine receptors on human colonic epithelial cells. The Journal of clinical investigation 104: 1061-1069.

Karagiannis P, Takahashi K, Saito M, Yoshida Y, Okita K, Watanabe A, et al. (2019). Induced Pluripotent Stem Cells and Their Use in Human Models of Disease and Development. Physiological Reviews 99: 79-114.

Kolios G, & Moodley Y (2013). Introduction to stem cells and regenerative medicine. Respiration 85: 3-10.

Kucia M, Halasa M, Wysoczynski M, Baskiewicz-Masiuk M, Moldenhawer S, Zuba-Surma E, et al. (2007). Morphological and molecular characterization of novel population of CXCR4+ SSEA-4+ Oct-4+ very small embryonic-like cells purified from human cord blood: preliminary report. Leukemia 21: 297-303.

Kucia M, Reca R, Campbell FR, Zuba-Surma E, Majka M, Ratajczak J, et al. (2006). A population of very small embryonic-like (VSEL) CXCR4(+)SSEA-1(+)Oct-4+ stem cells identified in adult bone marrow. Leukemia 20: 857-869.

Kuroda Y, Kitada M, Wakao S, Nishikawa K, Tanimura Y, Makinoshima H, et al. (2010). Unique multipotent cells in adult human mesenchymal cell populations. Proceedings of the National Academy of Sciences of the United States of America 107: 86398643.

Kuruca SE, Çelik DD, Özerkan D, & Erdemir G (2019). Characterization and Isolation of Very Small Embryonic-like (VSEL) Stem Cells Obtained from Various Human Hematopoietic Cell Sources. Stem Cell Rev Rep 15: 730-742.

Lee TC, Lee TH, Huang YH, Chang NK, Lin YJ, Chien PW, et al. (2014). Comparison of surface markers between human and rabbit mesenchymal stem cells. PloS one 9: e111390.

Malekmohammad K, Sewell RDE, & Rafieian-Kopaei M (2019). Antioxidants and Atherosclerosis: Mechanistic Aspects. Biomolecules 9.

Mao D, Ando S, Sato SI, Qin Y, Hirata N, Katsuda Y, et al. (2017). A Synthetic Hybrid Molecule for the Selective Removal of Human Pluripotent Stem Cells from Cell Mixtures. Angew Chem Int Ed Engl 56: 1765-1770.

McGrath KE, Koniski AD, Maltby KM, McGann JK, & Palis J (1999). Embryonic expression and function of the chemokine SDF-1 and its receptor, CXCR4. Developmental biology 213: 442-456.

Miller RJ, Banisadr G, & Bhattacharyya BJ (2008). CXCR4 signaling in the regulation of stem cell migration and development. Journal of neuroimmunology 198: 31-38.

Mo M, Wang S, Zhou Y, Li H, & Wu Y (2016). Mesenchymal stem cell subpopulations: phenotype, property and therapeutic potential. Cell Mol Life Sci 73: 33113321.

Monti M, Imberti B, Bianchi N, Pezzotta A, Morigi M, Del Fante C, et al. (2017). A Novel Method for Isolation of Pluripotent Stem Cells from Human Umbilical Cord Blood. Stem cells and development 26: 1258-1269.

Ntege EH, Sunami H, & Shimizu Y (2020). Advances in regenerative therapy: A review of the literature and future directions. Regen Ther 14: 136-153.

Pan G, & Thomson JA (2007). Nanog and transcriptional networks in embryonic stem cell pluripotency. Cell Res 17: 42-49.

Paspaliaris V, & Kolios G (2019). Stem cells in Osteoporosis: From Biology to New Therapeutic Approaches. Stem Cells Int 2019: 1730978.

Ratajczak MZ, Ratajczak J, Suszynska M, Miller DM, Kucia M, & Shin DM (2017). A Novel View of the Adult Stem Cell Compartment From the Perspective of a Quiescent Population of Very Small Embryonic-Like Stem Cells. Circulation research 120: 166-178.

Ratajczak MZ, Zuba-Surma E, Wojakowski W, Suszynska M, Mierzejewska K, Liu R, et al. (2014). Very small embryonic-like stem cells (VSELs) represent a real challenge in stem cell biology: recent pros and cons in the midst of a lively debate. Leukemia 28: 473-484.

Sato T, Wakao S, Kushida Y, Tatsumi K, Kitada M, Abe T, et al. (2020). A Novel Type of Stem Cells Double-Positive for SSEA-3 and CD45 in Human Peripheral Blood. Cell Transplant 29: 963689720923574.

Sauzay C, Voutetakis K, Chatziioannou A, Chevet E, & Avril T (2019). CD90/Thy-1, a Cancer-Associated Cell Surface Signaling Molecule. Front Cell Dev Biol 7: 66.

Shin DM, Liu R, Wu W, Waigel SJ, Zacharias W, Ratajczak MZ, et al. (2012). Global gene expression analysis of very small embryonic-like stem cells reveals that the Ezh2dependent bivalent domain mechanism contributes to their pluripotent state. Stem cells and development 21: 1639-1652.

Suman S, Domingues A, Ratajczak J, & Ratajczak MZ (2019). Potential Clinical Applications of Stem Cells in Regenerative Medicine. Adv Exp Med Biol 1201: 1-22. [0183] Takahashi K, Tanabe K, Ohnuki M, Narita M, Ichisaka T, Tomoda K, et al. (2007). Induction of pluripotent stem cells from adult human fibroblasts by defined factors. Cell 131: 861-872.

Takahashi K, & Yamanaka S (2006). Induction of pluripotent stem cells from mouse embryonic and adult fibroblast cultures by defined factors. Cell 126: 663-676.

Tarnok A, Ulrich H, & Bocsi J (2010). Phenotypes of stem cells from diverse origin. Cytometry A 77: 6-10.

Thomson JA, Itskovitz-Eldor J, Shapiro SS, Waknitz MA, Swiergiel JJ, Marshall VS, et al. (1998). Embryonic stem cell lines derived from human blastocysts. Science 282: 1145-1147.

Virant-Klun I, Zech N, Rozman P, Vogler A, Cvjeticanin B, Klemenc P, et al. (2008). Putative stem cells with an embryonic character isolated from the ovarian surface epithelium of women with no naturally present follicles and oocytes. Differentiation 76: 843856.

Wakao S, Kitada M, Kuroda Y, Shigemoto T, Matsuse D, Akashi H, et al. (2011). Multilineage-differentiating stress-enduring (Muse) cells are a primary source of induced pluripotent stem cells in human fibroblasts. Proceedings of the National Academy of Sciences of the United States of America 108: 9875-9880.

Ward SG, Bacon K, & Westwick J (1998). Chemokines and T lymphocytes: more than an attraction. Immunity 9: 1-11.

Wright AJ, & Andrews PW (2009). Surface marker antigens in the characterization of human embryonic stem cells. Stem Cell Res 3: 3-11.

Yang J, Song T, Wu P, Chen Y, Fan X, Chen H, et al. (2012). Differentiation potential of human mesenchymal stem cells derived from adipose tissue and bone marrow to sinus node-like cells. Mol Med Rep 5: 108-113.

Zhang Y, Kumagai K, & Saito T (2014). Effect of parathyroid hormone on early chondrogenic differentiation from mesenchymal stem cells. J Orthop Surg Res 9: 68.

Zuba-Surma EK, & Ratajczak MZ (2010). Overview of very small embryonic-like stem cells (VSELs) and methodology of their identification and isolation by flow cytometric methods. Curr Protoc Cytom Chapter 9: Unit9.29.

1. A method of isolating small blood stem cells (SBSCs) from peripheral blood, comprising:
centrifuging the peripheral blood to isolate plasma from the peripheral blood;
centrifuging the plasma to isolate a population of cells comprising the SBSCs, preferably in a cell pellet;
resuspending the population of cells comprising the SBSCs in a liquid; and
filtering the resuspended population of cells comprising the SBSCs through a filter having a pore size, which excludes cells that are larger than the SBSCs;
thereby isolating the SBSCs.

2-48. (canceled)

49. The method of claim 1, wherein the peripheral blood is centrifuged at a speed that is 200×g, 250×g, 300×g, 350×g, 400×g, 450×g, 500×g, 550×g, 600×g, 650×g, 700×g, 750×g, or 800×g, or about 200×g, about 250×g, about 300×g, about 350×g, about 400×g, about 450×g, about 500×g, about 550×g, about 600×g, about 650×g, about 700×g, about 750×g, or about 800×g, or any speed within a range defined by any two of the aforementioned speeds.

50. The method of claim 49, wherein the plasma is centrifuged at a speed that is 1000×g, 1050×g, 1100×g, 1150×g, 1200×g, 1250×g, 1300×g, 1350×g, or 1400×g, or about 1000×g, about 1050×g, about 1100×g, about 1150×g, about 1200×g, about 1250×g, about 1300×g, about 1350×g, or about 1400×g, or any speed within a range defined by any two of the aforementioned speeds.

51. The method of claim 50, wherein the pore size of the filter is 1 µm, 2 µm, 3 µm, 4 µm, 5 µm, 6 µm, 7 µm, 8 µm, 9 µm, 10 µm, 11 µm, 12 µm, 13 µm, 14 µm, 15 µm, 16 µm, 17 µm, 18 µm, 19 µm, or 20 µm, or about 1 µm, about 2 µm, about 3 µm, about 4 µm, about 5 µm, about 6 µm, about 7 µm, about 8 µm, about 9 µm, about 10 µm, about 11 µm, about 12 µm, about 13 µm, about 14 µm, about 15 µm, about 16 µm, about 17 µm, about 18 µm, about 19 µm, or about 20 µm, or any size within a range defined by any two of the aforementioned sizes, wherein cells that are larger than the pore size of the filter are excluded.

52. The method of claim 51, wherein the population of cells comprising the SBSCs is resuspended in an isotonic solution.

53. The method of claim 52, wherein the isotonic solution is growth medium, 0.9% saline, 5% dextrose solution, Ringer’s lactate solution, or Ringer’s acetate solution, or any combination thereof.

54. The method of claim 52, wherein the peripheral blood is mixed with an anticoagulant prior to centrifuging.

55. The method of claim 54, wherein the anticoagulant is sodium citrate.

56. The method of claim 54, wherein the population of cells comprising the SBSCs comprise cells having pluripotency markers, mesenchymal stem cell markers, or hematopoietic stem cell markers, or any combination thereof.

57. The method of claim 56, wherein the pluripotency markers comprise Nanog, Oct4, SOX-2, CXCR4, cMyc, KLF4, SSEA-3, or SSEA-4, or any combination thereof.

58. The method of claim 57, wherein the mesenchymal stem cell markers comprise CD29, PTH1R, CD105, or CD106, or any combination thereof.

59. The method of claim 58, wherein the hematopoietic stem cell markers comprise CD90, CD133, or CD45, or any combination thereof.

60. The method of claim 59, wherein the SBSCs stain with Kyoto Probe 1.

61. The method of claim 1, further comprising:
resuspending the isolated SBSCs in a cryopreservation medium;
freezing the SBSCs at -80° C.; and
transferring the frozen SBSCs to -150° C., and
wherein the cryopreservation medium is 10:1 human serum:DMSO, mFreSR, or Bambanker.

62. A method of treating or inhibiting chronic kidney disease in a subject in need thereof, comprising administering the isolated SBSCs of claim 1 to the subject.

63. The method of claim 62, wherein the SBSCs are allogeneic to the subject.

64. A method of treating or inhibiting osteoporosis in a subject in need thereof, comprising administering the SBSCs of claim 1 to the subject.

65. The method of claim 64, wherein the SBSCs are administered parenterally.

66. The method of claim 64, further comprising administering parathyroid hormone (PTH) to the subject.

67. The method of claim 64, wherein the SBSCs are allogeneic to the subject.
